(19) United States
(12) Patent Application Publication
(10) Pub. No.: US 2006/0264713 A1
(43) Pub. Date: Nov. 23, 2006

(54) DISEASE AND THERAPY DISSEMINATION REPRESENTATION

(76) Inventors: Christoph PEDAIN, Munich (DE); Andreas HARTLEP, Naring (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(21) Appl. No.: 11/419,535

(22) Filed: May 22, 2006

Related U.S. Application Data
(60) Provisional application No. 60/686,714, filed on Jun. 2, 2005.

(30) Foreign Application Priority Data
May 20, 2005 (EP) 05011039.4

(51) Int. Cl. G06G 7/48; A61B 5/00

(52) U.S. Cl. 600/300; 703/11

(57) ABSTRACT

A multi-layered representation and/or simulation of disease dissemination that may be complemented with consideration of therapy dissemination includes the creation of a multi-layered representation system that includes two or more of the following layers: a) a disease dissemination layer; b) a therapy dissemination layer; c) an interface layer; d) a dynamization layer; e) a solution layer; and f) a display layer.

### RELATED APPLICATION DATA

This application claims priority of U.S. Provisional Application No. 60/686,714 filed on Jun. 2, 2005, which is incorporated herein by reference in its entirety.

### FIELD OF THE INVENTION

The invention herein described relates to a multi-layered representation, modeling and/or simulation of disease dissemination that may be complemented with consideration of therapy dissemination.

### BACKGROUND OF THE INVENTION

For medical applications, it is desirable to determine the dissemination parameters and dissemination states of a disease. This task is diverse and can range from epidemiology to the local administration of therapies into a human or animal. The computational problem, however, usually is enormous, since it is not easy to capture the cross-relationship between the biological system, the disease dissemination, and actions taken to fight the disease and how these affect the disease dissemination, the disease, and the biological system.

Current approaches for the modeling of disease dissemination have relied on:
scenario based computations that do not or only partially include the dynamization possibility;
pattern recognition methods that refrain from an analytical description of disseminations;
neural networks, relying on training for pattern recognition and therefore lacking the ability to separate out interdependencies;
symptom focused disease identification methods, with the inability of taking into account the dynamic nature of the disease.
   For example, conventional modeling methods for radiation therapy, say in the case of brain tumors, allow the user to define risk structures (such as the optical nerve). The user then assigns a maximum tolerated radiation dose to that risk structure. This process is then repeated for multiple risk structures. In addition to defining such risk structures, the user also defines a desired radiation dose in the disease. Conventional radiation planning software then develops a treatment plan under consideration of the constraints imposed by the tolerated and desired radiation doses.

This approach, however, is limited in various respects. First, it only pertains to radiation therapy. The approach does not take into account that the application of other treatment methods to all or parts of the disease may be beneficial to achieve a better overall treatment effect. Second, this method of treatment planning neglects the dynamic behavior of the disease and does not take into account the brain tumor growth and spread patterns. Third, this method of treatment planning neglects the adverse effects that the radiation treatment has on the tumor spread patterns. For instance, radiation is known to cause tissue swelling (“edema”). Such edema results in a widening of the spaces between cells (“interstitial spaces”), hence making it easier for cancer cells to migrate away from the disease. Fourth, such method of treatment does not attempt to analyze the effect of the treatment onto the biological system, in the case of this example a brain cancer patient. There may be some side effects on the optical nerve (e.g., depending on the gravity of the disease in this area, the reversibility of the side effects, the occupational preference of the patient, and the like) that are tolerable,

U.S. Pat. No. 6,873,914 discloses methods and systems for analyzing complex biological systems. U.S. Pat. No. 6,882,990 discloses methods of identifying biological patterns using multiple data sets. U.S. Pat. No. 6,849,045 discloses computerized medical diagnostic and treatment advice system including network access. U.S. Pat. No. 6,789,069 discloses a method for enhancing knowledge discovered from biological data using a learning machine. U.S. Pat. No. 6,767,325 discloses an automated diagnostic system and method including synergies. U.S. Pat. No. 6,761,697 discloses methods and systems for predicting and/or tracking changes in external body conditions. U.S. Pat. No. 6,760,715 discloses enhancing biological knowledge discovery using multiple support vector machines. U.S. Pat. No. 6,746,399 discloses an automated diagnostic system and method including encoding patient data. U.S. Pat. No. 6,725,209 discloses a computerized medical diagnostic and treatment advice system and method including mental status examination. U.S. Pat. No. 6,714,925 discloses a system for identifying patterns in biological data using a distributed network. U.S. Pat. No. 6,617,114 discloses an identification of drug complementary combinatorial libraries. U.S. Pat. No. 6,597,996 discloses a method for identifying or characterizing properties of polymeric units. U.S. Pat. No. 6,569,093 discloses an automated diagnostic system and method including disease time line. U.S. Pat. No. 6,527,713 discloses an automated diagnostic system and method including alternative symptoms. U.S. Pat. No. 6,363,393 discloses a component based object-relational database infrastructure and user interface.

### SUMMARY OF THE INVENTION

The present invention enables use of multiple layers of information that are relevant for disease dissemination and therapy dissemination alike. A framework is provided in which relevant information can be efficiently stored, assessed, and used for subsequent simulations and computations. Moreover, there is provided an effective representation system that is suitable to distinguish relevant information about the disease, the therapy, the dynamic character of disease action and interaction, and the biological system. The framework can include the following features:
binding together (i.e., integrating) a variety of treatments;
dynamically predicting the effects of the disease on the biological system (e.g., a patient); and/or
dynamically predicting the effects of the treatment on both the biological system and the disease.

The invention proposes an analytical model that distinguishes the factors that determine a current state of a biological system or a disease from the factors that determine interrelations or dynamic behavior of both.

A method for multi-layered representation and/or simulation of disease dissemination is provided that may be complemented with consideration of therapy dissemination, and includes the creation of a multi-layered representation system that has two or more of the following layers:
a) a disease dissemination layer;
b) a therapy dissemination layer;
c) an interface layer;
d) a dynamization layer;
e) a solution layer; and
f) a display layer.

Further, one or more of the following creating activities can be carried out:
a. said disease dissemination layer can be created using the following steps:
   i. creating a model of disease dissemination;
   ii. identifying disease dissemination parameters;
   iii. extracting information about disease dissemination parameters from a biological system;
b. said therapy dissemination layer can be created using the following steps:
   i. creating a model of therapy dissemination;
   ii. identifying therapy dissemination parameters;
   iii. extracting information about therapy dissemination parameters from a therapy method;
c. said interface layer can be created using the following steps:
   i. extracting the cross-relationships between other layers from the biological system and/or the other layers;
   ii. identifying the cross influences that interface values have on the layers that incorporate the use of such interface value;
   iii. extracting information about interface values from either the disease, or the therapy method, or the biological system;
d. said dynamization layer can be created using the following steps:
   i. creating a model of dynamic response of one or more of the following to values of the interface layer: disease dissemination, therapy dissemination, disease state, therapy state, dissemination scenarios, the biological system, disease dissemination parameters, therapy dissemination parameters;
   ii. identifying dynamization parameters;
   iii. extracting dynamization parameters from a biological system;
e. said solution layer can be created using the following steps:
   i. including a timely term into the disease and therapy dissemination model;
   ii. solving for absolute state of disseminations at given time points;
f. said display layer can be created using one or more of the following steps:
   i. displaying or enhancing data that is relevant to or extracted from the biological system;
   ii. displaying or enhancing data that is relevant to the disease;
   iii. displaying or enhancing data that is relevant to the therapy;
   iv. displaying data that is relevant to the effect of the disease onto the biological system;
   v. displaying data that is relevant to the effect of the disease and the therapy onto the biological system.

At least one of said layers preferably is a collection of information, a database or a data processing program.

The dynamization layer may include the creation of boundary values describing distinguishable portions of the response of one of more of the following:
(a) the targeted biological system;
(b) the disease;
(c) the disease dissemination parameters;
(d) the therapy;
(e) the therapy dissemination parameters.

The solution layer may include or consist of separating the representation into a multitude of representations and separately solving each representation for the absolute state of disease and therapy disseminations.

Information used in the representations may include one or more of the following: prevalence, incidence, population, data acquired by magnetic resonance techniques (e.g., MRI, MRS, fMRI, MR-Perfusion Imaging, . . . ), computed tomography images, x-ray image data, SPECT-data, PET-data, data acquired by medical ultrasound techniques, other diagnostic medical data, age, average age, gender, habits, environmental conditions of said biological system, healthcare expenditure, per capita healthcare expenditure.

Information used in the representations may be co-registered with an individual subject. Then, the co-registration may include adaptation of the data to match the individual subject. In this case the adaptation may include deformation of data.

A multitude of diseases and their dissemination parameters can be represented. Also, a multitude of therapies and their dissemination parameters may be represented.

In one embodiment, only a subset of the layers is executed. The solution layer may include iterative execution of one or more layers with varying parameters.

In accordance with one embodiment of the invention, the display layer utilizes a computer screen to display a compounded image of at least two of the following: information about the biological system, information about the disease dissemination, information about the therapy dissemination, information about the effect of the disease on the biological system, information about the effect of the disease and the therapy on the biological system, therapy parameters, disease parameters, scenarios of representations, scenarios of solutions.

The information displayed about the biological system could be an image or graphical object computed from a medical imaging system. On the other hand, or in addition, the information displayed about one or more of the items except the biological system may be displayed in the form of objects overlaid onto the information displayed about the biological system.

The number of layers can be reduced by means of combination of layers. The method may be applied in a medical application. In this case, the medical application could be the identification of one or more disseminations within a human body. The disseminations might then be related to tumor cell migration and dissemination, in particular tumor cell migration concerning brain tumor cells.

In one embodiment, the layers are executed on a computer system and/or a network of computer systems with distributed tasks and databases.

The invention also provides a program which, when running on a computer or loaded into a computer, causes the computer to perform at least one of the methods described above. Moreover the invention provides a computer-program storage medium comprising such a program.

In another aspect, there is provided a device that may carry out at least one of the methods described herein. The device comprises at least one apparatus for multi-layered representation and/or simulation of disease dissemination that may be complemented with consideration of therapy dissemination, comprising the creation of a multi-layered representation system that includes two or more of the following layers: a disease dissemination layer; a therapy dissemination layer; an interface layer; a dynamization layer; a solution layer; and a display layer.

The device may comprise the following layers:
a. disease dissemination layer, which can be created using the following steps:
   i. creating a model of disease dissemination;
   ii. identifying disease dissemination parameters;
   iii. extracting information about disease dissemination parameters from a biological system;
b. therapy dissemination layer, which can be created using the following steps:
   i. creating a model of therapy dissemination;
   ii. identifying therapy dissemination parameters;
   iii. extracting information about therapy dissemination parameters from a therapy method;
c. interface layer, which can be created using the following steps:
   i. extracting the cross-relationships between other layers from the biological system and/or the other layers;
   ii. identifying the cross influences that interface values have on the layers that incorporate the use of such interface value;
   iii. extracting information about interface values from either the disease, or the therapy method, or the biological system;
d. dynamization layer, which can be created using the following steps:
   i. creating a model of dynamic response of one or more of the following to values of the interface layer: disease dissemination, therapy dissemination, disease state, therapy state, dissemination scenarios, the biological system, disease dissemination parameters, therapy dissemination parameters;
   ii. identifying dynamization parameters;
   iii. extracting dynamization parameters from a biological system;
e. solution layer, which can be created using the following steps:
   i. including a timely term into the disease and therapy dissemination model;
   ii. solving for absolute state of disseminations at given time points;
f. display layer, which can be created using one or more of the following steps:
   i. displaying or enhancing data that is relevant to or extracted from the biological system;
   ii. displaying or enhancing data that is relevant to the disease;
   iii. displaying or enhancing data that is relevant to the therapy;
   iv. displaying data that is relevant to the effect of the disease onto the biological system;
   v. displaying data that is relevant to the effect of the disease and the therapy onto the biological system.

To the accomplishment of the foregoing and related ends, the invention, then, comprises the features hereinafter fully described and particularly pointed out in the claims. The following description and the annexed drawings set forth in detail certain illustrative embodiments of the invention. These embodiments are indicative, however, of but a few of the various ways in which the principles of the invention may be employed.

### BRIEF DESCRIPTION OF THE DRAWINGS

The forgoing and other embodiments of the invention are hereinafter discussed with reference to the drawings.

FIG. 1 is a block diagram of an exemplary computer system that may be used to implement one or more methods in accordance with the invention.

### DETAILED DESCRIPTION

As used herein, the term “disease dissemination” is defined as the spread or progression of a disease, including actual and predicted spread or progression. The term “therapy dissemination” is defined as the effect that one or more therapies have on one or more diseases, including actual and predicted effects.

The invention enables a course of a disease and/or a course of treatment to be dynamically predicted using a priori information regarding the progress of the disease and/or treatment. For example, disease dissemination data, such as information regarding cell divisions per time unit, speed of migration, location of diseased tissue, stage of the disease, how the disease spreads throughout the body (e.g., pathways, nutrients), patient data (e.g., medical imaging, tests), etc., may be assembled to form a knowledge base of the disease and how it is expected to progress. Additionally, therapy dissemination data, such as effective tumor kill rate produced by radiation therapy, dose, delivery location of therapeutic agents, side effects of therapy, etc., may be assembled to form a knowledge base of one or more possible treatment results. The disease dissemination data and the therapy dissemination data then may be linked so as to enable the various therapeutic approaches to be dynamically evaluated with respect to the disease. Based on the evaluation, an optimal treatment plan may be selected. By analyzing the effects of a multitude of treatment therapies, the subsequent progression of the disease can be predicted. This enables the physician to better combine various treatment therapies to optimally treat the disease.

An implementation of the invention will now be described with respect to a specific example wherein a patient has been diagnosed with a Glioblastoma Multiforme, a primary brain tumor that grows very fast and proliferates very swiftly. The model would be applied as follows:

A disease dissemination layer would be created. This layer would describe:
the rate of cell division per time unit, and the speed of cancer cell migration. Also, this layer would include the effects on normal cell population that a certain density of cancer cells per tissue volume would have.
the pathways of cancer cell migration, e.g., a preference of spread along white matter tracks in the brain. Further, a dependency on the size of the interstitial space (“pore fraction”)
the source data for all necessary information, e.g., literature values for the rate of cell division and the basic speed of cancer cell migration, or diffusion tensor MRI scans for obtaining the pathways of cancer cell spread, or multiple b value diffusion tensor MRI scans for determining the local variations of the size of the interstitial spaces.

A therapy dissemination layer would be created. This layer would describe:
a radiation dose distribution model, e.g., a spatial map of dose levels created by an external beam radiation therapy. Also, this layer would describe the effects on cancer cell population that a certain dose level would have in a fraction of tissue volume, and the effects on edema and on normal cell population within this volume.
an adjustment of the radiation therapy based on the local variation of tissue densities.
the source data for all necessary information, e.g., literature values for the kill rate of a certain cell type dependant on a certain radiation dose, or CT scans for density.

An interface layer would be created. This layer would contain the information that describes the interrelation between the disease and the therapy. In the example, this would be:
for the biological system (the patient), a side effect measure dependent on normal cell killing. This side effect ratio would be dependent on the region of the brain where the normal cell kill occurs—e.g., killing a certain fraction of the optical nerve or the motor area causes a severe side effect, whereas that same tissue fraction kill in a different area of the brain may be less harmful.
for the disease, the cell population parameters as mentioned above. Also, for the disease, the adverse effect a certain population of cancer cells per volume has on the survival of normal cells within that same volume.
for the treatment, the effects on survival of both normal and cancer cells.
   A mathematical formula can be used to link the above parameters with one another.

A dynamization layer would be created, including:
for the disease, the speed of cancer cell migration with a dependency on the size of the interstitial space and the nerve fiber directions.
for the therapy, the effects of a certain dose on the size of the interstitial space, in a time and distance dependent manner.
for the biological system, the position of the nerve fiber tracks.
for the edema, the dependency of the edema spread to nerve fibers.
a mathematical formula that links the above parameters with one another.

A solution layer would be created, containing:
a mathematical formula that links the dissemination layers with the dynamization layer.
a solution method, e.g., a numerical method, that optimizes a delivery pattern (radiation dose, fractionation) regarding the side effects created by the therapy and disease dissemination.

Finally, a display layer would create graphic representations of the various clinical options that are developed in the solution layer.

This example is already a significant improvement over existing radiation therapy approaches, since now the patient specific effects and side effects can be regarded in a holistic manner.

A benefit of the proposed method, however, comes into play when various different therapies are linked with one another. Say we have a therapy that can treat GBM cells but is largely selective and does not affect healthy brain cells in the same gravity radiation would. The systematic model above now allows to include this method into the treatment optimization model, since the underlying pattern is identical: This additional treatment model again has some effect on the brain cancer cells, and some effect on the healthy cells, which means it can be easily included into the solution layer.

In fact, every therapy against GBM can follow this underlying pattern and may be added to the optimization method.

In summary, the method allows a generic and much improved manner to plan for treatments, whether it pertains to a single treatment, or to a combination of treatments.

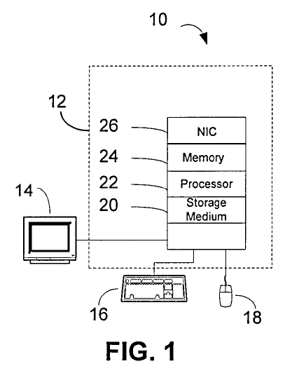

FIG. 1 is a block diagram of a system 10 for implementing one or more of the methods described herein. The system 10 includes a computer 12 for processing data, and a display 14 for viewing system information. The technology used in the display is not critical and may be any type currently available, such as a flat panel liquid crystal display (LCD) or a cathode ray tube (CRT) display, or any display subsequently developed. A keyboard 16 and pointing device 18 may be used for data entry, data display, screen navigation, etc. The keyboard 16 and pointing device 18 may be separate from the computer 12 or they may be integral to it. A computer mouse or other device that points to or otherwise identifies a location, action, etc., e.g., by a point and click method or some other method, are examples of a pointing device. Alternatively, a touch screen (not shown) may be used in place of the keyboard 16 and pointing device 18. A touch screen is well known by those skilled in the art and will not be described in detail herein. Briefly, a touch screen implements a thin transparent membrane over the viewing area of the display 14. Touching the viewing area sends a signal to the computer 12 indicative of the location touched on the screen. The computer 12 may equate the signal in a manner equivalent to a pointing device and act accordingly. For example, an object on the display 14 may be designated in software as having a particular function (e.g., view a different screen). Touching the object may have the same effect as directing the pointing device 18 over the object and selecting the object with the pointing device, e.g., by clicking a mouse. Touch screens may be beneficial when the available space for a keyboard 16 and/or a pointing device 78 is limited.

Included in the computer 12 is a storage medium 20 for storing information, such as application data, screen information, programs, etc., which may be in the form of a database 21. The storage medium 20 may be a hard drive, for example. A processor 22, such as an AMD Athlon 64® processor or an Intel Pentium IV® processor, combined with a memory 24 and the storage medium 20 execute programs to perform various functions, such as data entry, numerical calculations, screen display, system setup, etc. A network interface card (NIC) 26 allows the computer 22 to communicate with devices external to the system 10.

The actual code for performing the functions described herein can be readily programmed by a person having ordinary skill in the art of computer programming in any of a number of conventional programming languages based on the disclosure herein. Consequently, further detail as to the particular code itself has been omitted for sake of brevity.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for multi-layered representation and/or simulation of disease dissemination that may be complemented with consideration of therapy dissemination, comprising the creation of a multi-layered representation system that includes two or more of the following layers:
a) a disease dissemination layer;
b) a therapy dissemination layer;
c) an interface layer;
d) a dynamization layer;
e) a solution layer; and
f) a display layer.

2. The method of claim 1, wherein one or more of the following creating activities is/are carried out:
g. said disease dissemination layer is created using the following steps:
i. creating a model of disease dissemination;
ii. identifying disease dissemination parameters;
iii. extracting information about disease dissemination parameters from a biological system;
h. said therapy dissemination layer is created using the following steps:
i. creating a model of therapy dissemination;
ii. identifying therapy dissemination parameters;
iii. extracting information about therapy dissemination parameters from a therapy method;
i. said interface layer is created using the following steps:
i. extracting the cross-relationships between other layers from the biological system and/or the other layers;
ii. identifying the cross influences that interface values have on the layers that incorporate the use of such interface value;
iii. extracting information about interface values from either the disease, or the therapy method, or the biological system;
j. said dynamization layer is created using the following steps:
i. creating a model of dynamic response of one or more of the following to values of the interface layer: disease dissemination, therapy dissemination, disease state, therapy state, dissemination scenarios, the biological system, disease dissemination parameters, therapy dissemination parameters;
ii. identifying dynamization parameters;
iii. extracting dynamization parameters from a biological system;
k. said solution layer is created using the following steps:
i. including a timely term into the disease and therapy dissemination model;
ii. solving for absolute state of disseminations at given time points;
l. said display layer is created using one or more of the following steps:
i. displaying or enhancing data that is relevant to or extracted from the biological system;
ii. displaying or enhancing data that is relevant to the disease;
iii. displaying or enhancing data that is relevant to the therapy;
iv. displaying data that is relevant to the effect of the disease onto the biological system;
v. displaying data that is relevant to the effect of the disease and the therapy onto the biological system.

3. The method of claim 1, wherein at least one of said layers is a collection of information, a database or a data processing program.

4. The method of claim 1, wherein the dynamization layer includes the creation of boundary values describing distinguishable portions of the response of one of more of the following:
(a) the targeted biological system,
(b) the disease,
(c) the disease dissemination parameters,
(d) the therapy,
(e) the therapy dissemination parameters.

5. The method of claim 1, wherein the solution layer includes separating the solution layer into a multitude of representations and separately solving each representation for the absolute state of disease and therapy disseminations.

6. The method of claim 1, wherein information used in the representations includes one or more of the following: prevalence, incidence, population, data acquired by magnetic resonance techniques, computed tomography images, x-ray image data, SPECT-data, PET-data, data acquired by medical ultrasound techniques, other diagnostic medical data, age, average age, gender, habits, environmental conditions of said biological system, healthcare expenditure, or per capita healthcare expenditure.

7. The method of claim 1, wherein information used in the representations is co-registered with a individual subject.

8. The method of claim 7, wherein the co-registration includes adaptation of the data to match the individual subject.

9. The method of claim 8 wherein the adaptation includes deformation of data.

10. The method of claim 1, wherein a multitude of diseases and their dissemination parameters are represented.

11. The method of claim 1, wherein a multitude of therapies and their dissemination parameters are represented.

12. The method of claim 1, wherein only a subset of the layers are executed.

13. The method of claim 1, wherein the solution layer includes iterative execution of one or more layers with varying parameters.

14. The method of claim 1, wherein the display layer utilizes a computer screen to display a compounded image of at least two of the following: information about the biological system, information about the disease dissemination, information about the therapy dissemination, information about the effect of the disease on the biological system, information about the effect of the disease and the therapy on the biological system, therapy parameters, disease parameters, scenarios of representations, scenarios of solutions.

15. The method of claim 14, wherein the information displayed about the biological system is an image and/or a graphical object computed from a medical imaging system.

16. The method of claim 14, wherein the information displayed about one or more of the items except the biological system are displayed in the form of objects overlaid onto the information displayed about the biological system.

17. The method of claim 1, wherein the number of layers is reduced by combining layers.

18. The method of claim 1, wherein the method is applied in a medical application.

19. The method of claim 18, wherein the medical application is the identification of one or more disseminations within a human body.

20. The method of claim 19, wherein the disseminations are related to tumor cell migration and dissemination.

21. The method of claim 20, wherein the tumor cell migration concerns brain tumor cells.

22. The method of claim 1, wherein the layers are executed on a computer system and/or a network of computer systems with distributed tasks and databases.

23. A computer program embodied on a computer readable medium for multi-layered representation and/or simulation of disease dissemination that may be complemented with consideration of therapy dissemination, comprising code that creates a multi-layered representation system that includes two or more of the following layers:
a) a disease dissemination layer;
b) a therapy dissemination layer;
c) an interface layer;
d) a dynamization layer;
e) a solution layer; and
f) a display layer.

24. A device for multi-layered representation and/or simulation of disease dissemination that may be complemented with consideration of therapy dissemination, comprising:
a processor circuit including a processor and memory; and
logic stored in the memory and executed by the processor to create a multi-layered representation system that includes two or more of the following layers:
a) a disease dissemination layer;
b) a therapy dissemination layer;
c) an interface layer;
d) a dynamization layer;
e) a solution layer; and
f) a display layer.

25. The device of claim 24 comprising the following layers:
m. disease dissemination layer, which is created using the following steps:
i. creating a model of disease dissemination;
ii. identifying disease dissemination parameters;
iii. extracting information about disease dissemination parameters from a biological system;
n. therapy dissemination layer, which is created using the following steps:
i. creating a model of therapy dissemination;
ii. identifying therapy dissemination parameters;
iii. extracting information about therapy dissemination parameters from a therapy method;
o. interface layer, which is created using the following steps:
i. extracting the cross-relationships between other layers from the biological system and/or the other layers;
ii. identifying the cross influences that interface values have on the layers that incorporate the use of such interface value;
iii. extracting information about interface values from either the disease, or the therapy method, or the biological system;
p. dynamization layer, which is created using the following steps:
i. creating a model of dynamic response of one or more of the following to values of the interface layer: disease dissemination, therapy dissemination, disease state, therapy state, dissemination scenarios, the biological system, disease dissemination parameters, therapy dissemination parameters;
ii. identifying dynamization parameters;
iii. extracting dynamization parameters from a biological system;
q. solution layer, which is created using the following steps:
i. including a timely term into the disease and therapy dissemination model;
ii. solving for absolute state of disseminations at given time points;
r. display layer, which is created using one or more of the following steps:
i. displaying or enhancing data that is relevant to or extracted from the biological system;
ii. displaying or enhancing data that is relevant to the disease;
iii. displaying or enhancing data that is relevant to the therapy;
iv. displaying data that is relevant to the effect of the disease onto the biological system;
v. displaying data that is relevant to the effect of the disease and the therapy onto the biological system.

26. A method for predicting a disease progression and effects one or treatment therapies have on the disease progression, comprising:
linking disease dissemination data with therapy dissemination data; and
creating a simulation of the disease progression based on the linked data.

27. The method of claim 26, wherein creating the simulation includes creating a dynamic simulation so as to enable one or more treatment therapies to be evaluated with respect to disease progression.

28. The method of claim 27, further comprising performing the simulation in real time.

29. The method of claim 26, further comprising displaying the simulation results.

30. The method of claim 26, further comprising providing one or more optimal treatments for the disease based on patient criteria.

31. A method for predicting a disease progression, comprising:
assembling a priori information relating to one or more diseases;
assembling patient specific data; and
linking the patient specific data to a priori information.

32. The method of claim 31, wherein linking includes extracting cross-relationships between a priori information and the patient specific data.
